SEQUENCE LISTING (2 sequences)

SEQ ID NO: 1 - protein, 125 aa, Apis mellifera
KNGIYGIALSPVTNNLYYSPLLSHGLYYVDTEQFSNPQYEENNVQYEGSQDILNTQSFGKVVSKNGVLFLGLVGNSGIACVNEHQVLQRESFDVVAQNEETLQMIVSMKIMENLPQSGRINDPEG

SEQ ID NO: 2 - protein, 544 aa, Apis mellifera
MTKWLLLVVCLGIACQDVTSAAVNHQRKSANNLAHSMKVIYEWKHIDFDFGSDERRDAAIKSGEFDHTKNYPFDVDRWRDKTFVTIERNNGVPSSLNVVTNKKGKGGPLLRPYPDWSFAKYEDCSGIVSAFKIAVDKFDRLWVLDSGLVNNNQPMCSPKLLTFDLKTSKLVKQVEIPHNIAVNATTGMGELVSLAVQAIDRTNTMVYIADEKGEGLIMYQNSDDSFHRLTSNTFDYDPRYTKLTVAGESFTVKNGIYGIALSPVTNNLYYSPLLSHGLYYVDTEQFSNPQYEENNVQYEGSQDILNTQSFGKVVSKNGVLFLGLVGNSGIACVNEHQVLQRESFDVVAQNEETLQMIVSMKIMENLPQSGRINDPEGNEYMLALSNRMQKIINNDFNFNDVNFRILGANVDDLMRNTRCGRYHNQNAGNQNADNQNADNQNANNQNADNQNANKQNGNRQNDNRQNDNKQNGNRQNDNKQNGNRQNDNKQNGNRQNGNKQNDNKQNGNRQNDNKRNGNRQNDNQNNQNDNNRNDNQVHHSSKLH

(19) United States
(12) Patent Application Publication
(10) Pub. No.: US 2022/0280605 A1
(43) Pub. Date: Sep. 8, 2022

(54) A Composition And Method For Improving The Profile Of Blood

(71) Applicant: Manukamed Functional Foods Ltd., Masterton (NZ)

(72) Inventors: Denis Watson, Opaki (NZ); Christopher McMahon, Hamilton (NZ); Blake Paget, Hamilton (NZ); David Norrie, Auckland (NZ); Kim Whiteman, Pirongia (NZ); Frank Sieg, Northland (NZ)

(21) Appl. No.: 17/607,989

(22) PCT Filed: Apr. 29, 2020
(86) PCT No.: PCT/NZ2020/050039

§ 371 Date: Nov. 1, 2021

(30) Foreign Application Priority Data
May 1, 2019 (NZ) 753068

(51) Int. Cl. A61K 38/17; A61K 35/644; A61P 3/06; A61P 3/08

(52) U.S. Cl. CPC A61K 38/1767; A61K 35/644; A61P 3/06; A61P 3/08

(57) ABSTRACT

Use of a peptide or fragment thereof comprising SEQ ID NO. 1, or a peptide or fragment having at least 80% sequence identity thereto, in the manufacture of a medicament for one or more of reducing triglycerides, reducing LDL, reducing sdLDL, reducing glucose, reducing insulin, reducing HbA1c and increasing insulin sensitivity.

### FIELD OF INVENTION

This invention relates to a product and method for improving the blood profile of mammals, especially humans, for enhanced health.

### BACKGROUND

Having a good blood profile is important for healthy living and lifespan.

There are nine major royal jelly proteins (numbered MRJP1-9) in royal jelly and in honey (Buttstedt et al., 2014). They share 111 conserved amino acids and have sequence identity ranging between 47 and 74% (Buttstedt et al., 2014). MRJP3 is a 544 amino acid peptide that has a 20 amino acid signal peptide, making it 522 amino acids in length after secretion (Schmitzova et al., 1998). During digestion, MRJP3 is cleaved into multiple amino acid fragments, the longest of which are 400 amino acids (aa 21-421) and the C-terminal 122 amino acids (aa 422-544). They are stable for about an hour before being further digested into smaller fragments (Muresan et al., 2018). This pattern of cleavage is consistent with the C-terminal peptide providing a source of nitrogen (the region has repeat amino acid motifs rich in nitrogen) for rearing developing larvae (Albertova et al., 2005).

The inventors have ascertained that at least certain fragments of MRJP3 assist with maintaining good blood health.

### OBJECT

It is an object of a preferred form of the invention to go at least some way towards providing for improved blood profile of humans. However it should be appreciated that the object of the invention per se is simply to provide the public with a useful choice.

### DEFINITIONS

The term “comprises” or “has”, if and when used in this document in relation to one or more features, should not be seen as excluding the option of there being additional unspecified features (the features may for example be physical parts and/or action steps). The same definition applies to derivative terms such as “comprising” and “having”.

### SUMMARY OF THE INVENTION

In one aspect the invention involves the use of a peptide or fragment thereof (e.g. the peptide or fragment may or may not be part of a longer chain) comprising SEQ ID NO. 1* in the manufacture of a medicament for one or more of: *SEQ ID NO. 1 is shown in FIG. 2.

a) reducing triglycerides;

b) reducing LDL;

c) reducing sdLDL;

d) reducing glucose;

e) reducing insulin;

f) reducing MRJP1c; and

g) increasing insulin sensitivity.

Optionally the SEQ ID NO. 1 is within MRJP3.

Optionally the SEQ ID NO. 1 is within Honey (eg Manuka Honey).

According to another aspect, the invention is a method of treatment, comprising administering or taking a peptide or fragment thereof, comprising SEQ ID NO. 1 (e.g. the peptide or fragment may or may not be part of a longer chain) to:

a) reduce triglycerides;

b) reduce LDL;

c) reduce sdLDL;

d) reduce glucose;

e) reduce insulin;

f) reduce HbA1c; and g) increase insulin sensitivity.

Optionally the SEQ ID NO. 1 is within MRJP3.

According to a further aspect, the invention comprises the use of MRJP3, or honey containing MRJP3, in the manufacture of a medicament for one or more of:

a) reducing triglycerides;

b) reducing LDL;

c) reducing sdLDL;

d) reducing glucose;

e) reducing insulin;

f) reducing HbA1c; and

g) increasing insulin sensitivity.

According to another aspect, the invention is a method of treatment comprising administering or taking MRJP3, or honey containing MRJP3, to:

a) reduce triglycerides;

b) reduce LDL;

c) reduce sdLDL;

d) reduce glucose;

e) reduce insulin;

f) reduce HbA1c; and

g) increase insulin sensitivity.

All of the above may be in relation to humans or non-human animals.

According to further aspects of the invention the peptide or fragment mentioned above may in each case comprise or consist of 50-99% (preferably at least 80%, 85%, 90% or 95%, and most preferably 99%-100%) sequence identity to SEQ ID NO 1.

Preferably all the aspects and embodiments of the invention relate to medicaments or methods for treating humans. However in some instances they may be used for non-human mammals.

### DRAWINGS

FIG. 1 illustrates the amino acid sequence of MRJP3;

FIG. 2 illustrates the amino acid sequence of a particular 125mer sequence within MRJP3;

FIG. 3 graphically illustrates the growth rate (A) and body mass (B) of mice that were injected with the 125mer (the Injection Trial);

FIG. 4 graphically illustrates blood insulin (A) and glucose (B) levels of mice in the Injection Trial;

FIG. 5 graphically indicates insulin sensitivity of mice in the Injection Trial;

FIG. 6 graphically indicates total cholesterol levels of mice in the Injection Trial;

FIG. 7 graphically indicates HDL (A) and LDL (B) cholesterol levels of mice in the Injection Trial;

FIG. 8 graphically indicates the heart mass of mice in the Injection Trial;

FIG. 9 graphically indicates the arterial pressure (A) and heart rate (B) of mice in the Injection Trial;

FIG. 10 graphically illustrates the growth rate (A) and body mass (B) of mice that ingested manuka honey (the Honey Trial);

FIG. 11 graphically illustrates the mass of fat of mice in the Honey Trial;

FIG. 12 graphically illustrates blood glucose (A) and insulin (B) levels of mice in the Honey Trial;

FIG. 13 graphically illustrates blood HbA1c levels of mice in the Honey Trial;

FIG. 14 graphically illustrates blood insulin sensitivity of mice in the Honey Trial;

FIG. 15 graphically illustrates blood HDL (A) and LDL (B) cholesterol levels of mice in the Honey Trial;

FIG. 16 graphically illustrates blood sdLDL (A) and IbLDL (B) cholesterol levels of mice in the Honey Trial;

FIG. 17 graphically illustrates blood sdLDL cholesterol levels of mice in the Honey Trial;

FIG. 18 graphically illustrates blood triglyceride levels (A) and the ratio of triglyceride (TAG) to HDL (B) in blood of mice in the Honey Trial;

FIG. 19 graphically illustrates blood lipoprotein lipase activity (A) and blood VLDL levels (B) of mice in the Honey Trial;

FIG. 20 graphically illustrates blood ApoB levels (A) and the ratio of LDL to ApoB (B) in blood of mice;

FIG. 21 graphically illustrates the blood pressure of mice in the Honey Trial;

FIG. 22 graphically illustrates the intake of food and water of mice in the Honey Trial; and

FIG. 23 schematically illustrates a method of enriching honey with royal jelly.

### DETAILED DESCRIPTION

The invention is based on the discovery that that mice injected subcutaneously with a 125mer truncated peptide from MRJP3, or mice ingesting manuka honey incorporating the 125mer within MRJP3, have:

a) reduced blood concentrations of:
i) triglycerides;
ii) LDL and/or small dense LDL (sdLDL) cholesterol;
iii) insulin;
iv) glucose
v) HbA1c; and

b) increased insulin sensitivity.

It was also discovered that obese, diabetic mice (Lepr^{db}) injected with either a 125mer truncated peptide from MRJP3, or ingesting manuka honey incorporating the 125mer within MRJP3 have:

c) reduced adiposity;

d) lower blood pressure;

e) reduced cardiomyopathy; and

f) improved cardiac performance.

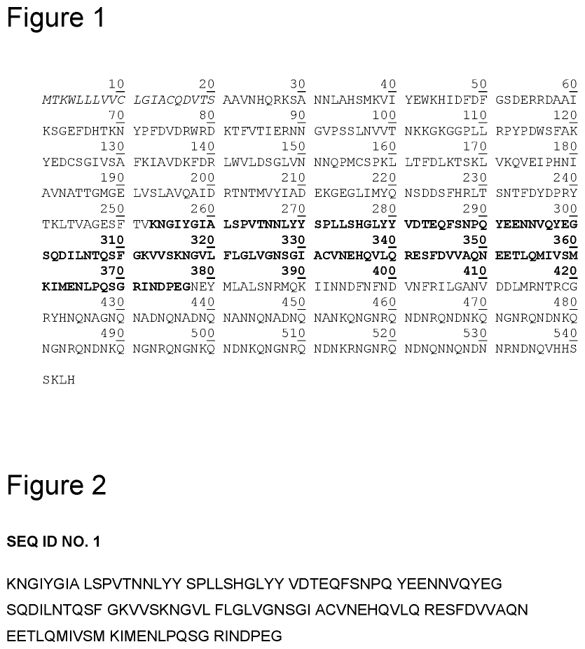

All the above discoveries came out of trials where recombinant peptides of various lengths were produced within the 400 amino acid N-terminal peptide region of MRJP3 that includes the most divergent region relative to the other 8 MRJP proteins (aa 282-311) as shown in FIG. 1. They were tested to assess their impact on the blood profile of mice, or on expression of genes from cultured cells in vitro.

To elaborate, FIG. 1 shows MRJP3 and the peptide fragments tested. The signal peptide is shown in italics. A C-terminal truncated protein was made (amino acids 21 to 420). Peptides of this truncated protein were made to an N-terminal region (amino acids 21 to 217), a middle-peptide (amino acids 119 to 319), a C-terminal peptide (amino acids 218 to 420). FIG. 2 shows the amino acid sequence of the 125mer peptide (aa 253-377) (SEQ ID NO. 1), namely—

KNGIYGIA LSPVTNNLYY SPLLSHGLYY VDTEQFSNPQ
YEENNVQYEG SQDILNTQSF GKVVSKNGVL FLGLVGNSGI
ACVNEHQVLQ RESFDVVAQN EETLQMIVSM KIMENLPQSG
RINDPEG

1st Trial—Administration by Injection

Male wild-type (C57BL/6J, stock number 000664) and Lepr^{db} mice (stock number 000697) were obtained at 4-5 weeks of age from The Jackson Laboratory (Bar Harbor, Me. USA). This strain has a naturally occurring mutation in the leptin receptor and they develop insulin resistance, impaired glucose tolerance and type 2 diabetes by 8 weeks of age.

Mice were housed with companions (3 to 4 per cage) in a temperature controlled (21° C.) and light controlled (12 h:12 h, light: dark cycle) animal facility at Ruakura Research Centre, Hamilton, New Zealand. Food (rodent chow) and water were available ad libitum.

Mice were randomly allocated to receive either vehicle (sterile saline) or 200 μg of discrete 125mer obtained from MRJP3 protein , injected subcutaneously every second day for 12 weeks from 6 to 18 weeks of age (n=10 per group).

Blood pressure was monitored at a week before death using a tail-cuff apparatus (BP-2000, Visitech Systems).

The mice were killed by CO₂ asphyxiation followed by cervical dislocation at 18 weeks and, at death, a blood sample was collected via cardiac puncture. Glucose was measured by glucometer (ACCU-CHEK Performa, Roache Diagnostics, USA). The mass of fat depots in the abdomen were weighed. Plasma was harvested and concentrations of insulin, HbA1c, triglycerides, total cholesterol and LDL/HDL cholesterol were assayed using commercial kits (Sigma Aldrich, Auckland, New Zealand and Crystal Chem, IL, USA).

Lower Growth Rate and Body Mass (by Injection)

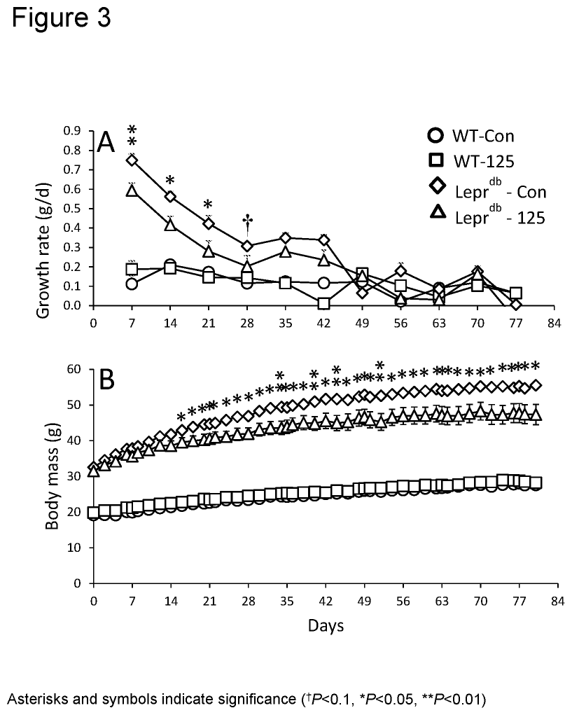

The Lepr^{db} mice that were given the 125mer were found to have a reduced rate of growth. As illustrated in FIG. 3, on average they had 13% less body mass at maturity. Reduced rate of growth from childhood and body mass in obese subjects are markers for reduced adiposity, which is an important risk factor in cardiovascular disease in both human (Bjerregaard et al 2019) and non-human animals.

Insulin & Glucose (by Injection)

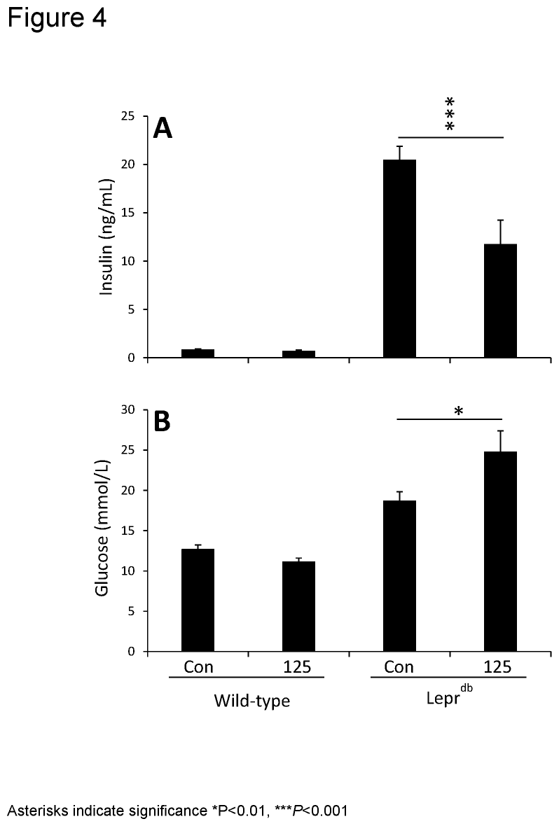

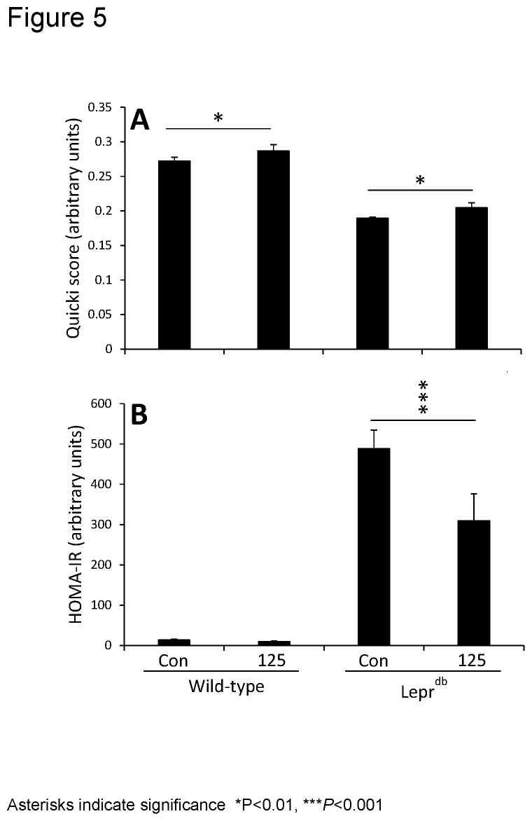

After 12 weeks of treatment the 125mer mice were also found to have a lower concentration of insulin, with an increased glucose concentration. This is illustrated in FIG. 4. Despite the increase in glucose, sensitivity to insulin was found to have increased, as assessed by way of the Quicki and HOMA-IR indices (Matthews et al., 1985, Katz et al., 2000). This is illustrated in FIG. 5. Reduced insulin and increased insulin sensitivity are markers for improvement in glucose homeostasis and a reduced risk of type II diabetes mellitus in human and non-human animals (Matthews et al., 1985, Katz et al., 2000)

Reduced Cholesterol (by Injection)

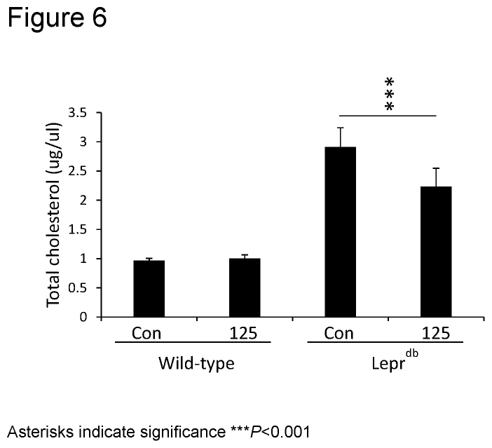

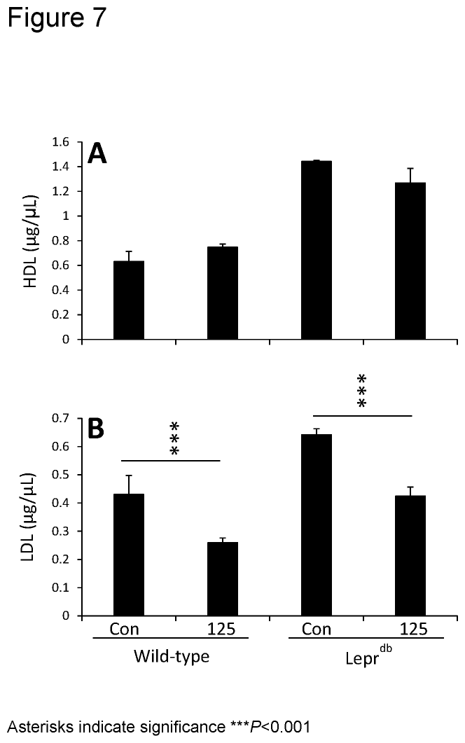

It was also found that the 125mer mice had reduced concentrations of total cholesterol, by about 30% (FIG. 6), and reduced concentrations of LDL cholesterol by up to 40% in lean, control and obese, diabetic mice (FIG. 7B) without change to the concentration of HDL cholesterol (FIG. 7A). Reduced total and LDL cholesterol, without a change in HDL cholesterol, are markers for a reduced risk of cardiovascular disease in human and non-human animals.

Reduced Arterial Pressure & Heart Rate (by injection)

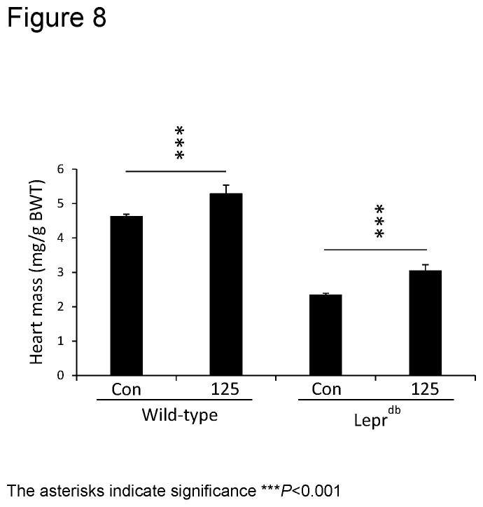

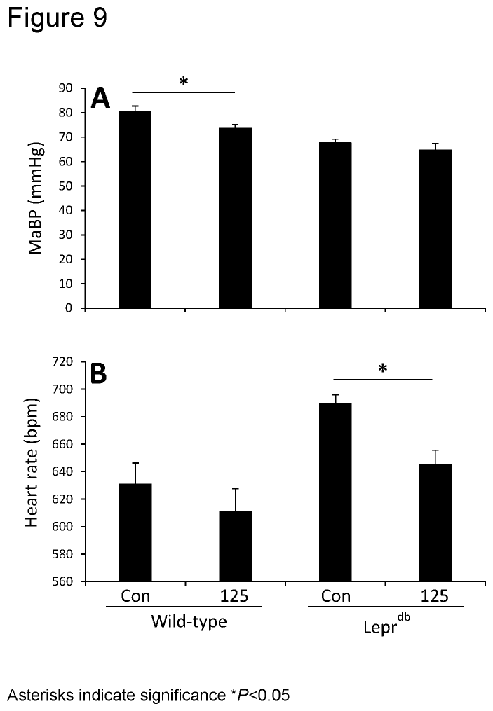

Lepr^{db} mice are in general susceptible to cardiomyopathy after 8-10 weeks of age, which reduces cardiac function and induces tachycardia to compensate for the reduced output (Belke and Severson, 2012). However, it was found that the mice given the 125mer had an increased heart mass, in both wild-type and Lepr^{db} mice (FIG. 8). This was coupled with reduced mean arterial pressure and reduced heart rate (FIG. 9). Increased heart mass, reduced arterial pressure and reduced heart rate in obese subjects are markers for a reduced risk of cardiovascular disease in human and non-human animals.

2^{nd} Trial—Administration by Ingestion

A second trial was run to test whether oral consumption of manuka honey as a source of the 125mer peptide would give the same results as administration by injection as above. This involved feeding manuka honey to obese male mice, Lepr^{db}, stock number 000697, from The Jackson Laboratory (Bar Harbor, Me. USA). Mice were allocated at random to have honey provided at 0%, 2.5%, 5% and 10% w/v in drinking water for 8 weeks, from 6 weeks of age (n=10 per group). The Control (Con) was water

The mice were housed 2-3 per cage in a temperature controlled (21° C.) and light controlled (12 h:12 h, light: dark cycle) animal facility at Ruakura Research Centre, Hamilton, New Zealand. The mice had ad libitum access to a standard rodent chow and water/manuka honey water. They were weighed weekly. Blood was collected at death via cardiac puncture and measurements and assays were carried as per the first experiment.

Lower Growth Rate and Body Mass (by Ingestion)

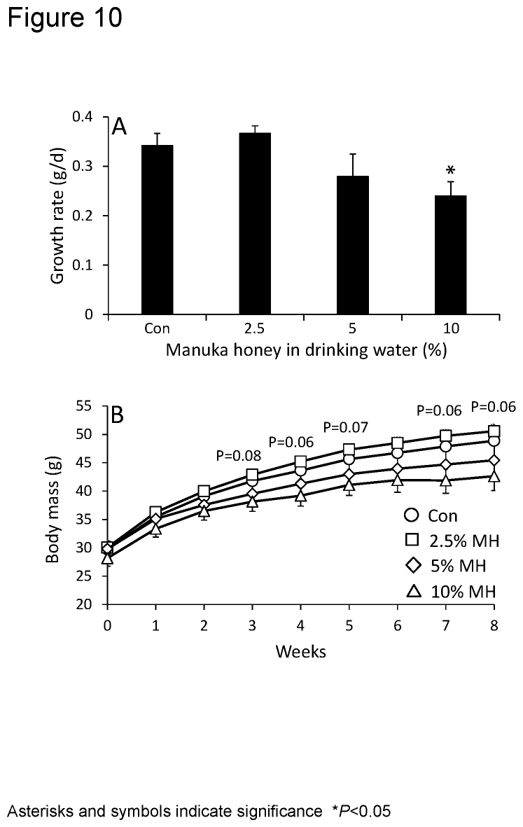

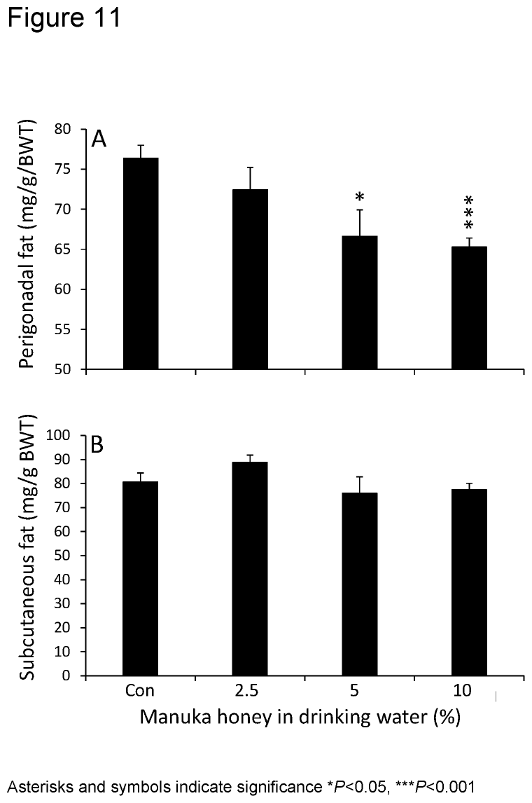

There was found to be a dose-dependent response rate for manuka honey, in other words an increasing dose corresponded with a reduced rate of growth (FIG. 10A) and body mass (FIG. 10B). Further, the mass of perigonadal fat (FIG. 11A), but not subcutaneous fat (FIG. 11B), reduced in a dose-dependent manner. The mass of perigonadal fat is a proxy for total body fat (Rogers and Webb, 1980). It was concluded that manuka honey at 5% and 10% in drinking water reduced adiposity.

Insulin & Glucose (by Ingestion)

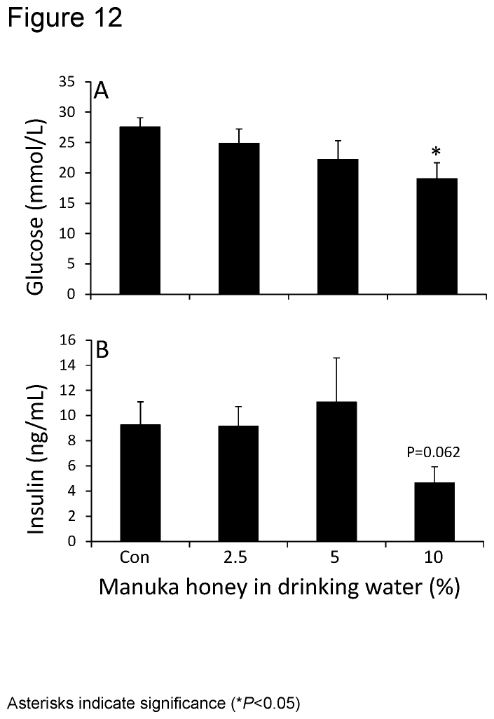

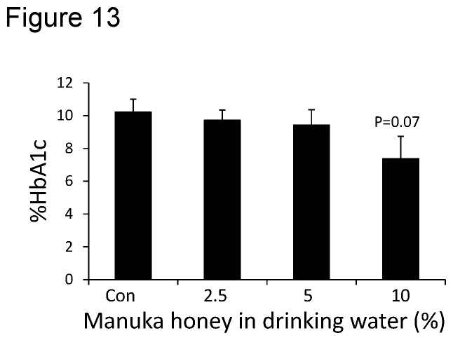

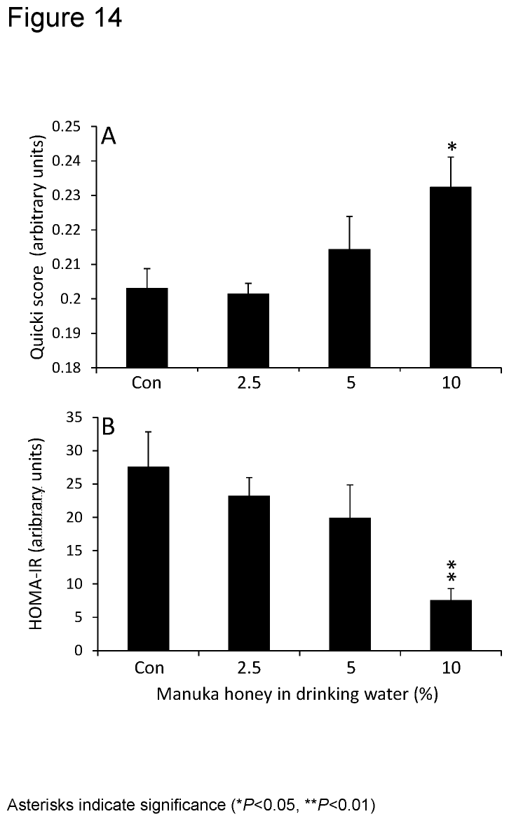

For the oral administration, concentrations of glucose and insulin were also reduced (FIG. 12), as were concentrations of HbA1c (FIG. 13), which is a marker of longer-term changes in glucose over 2-3 months (Kovatchev, 2017). Insulin sensitivity was also improved, as assessed using the Quicki and HOMA-IR indices (FIG. 14).

Cholesterol (by Ingestion)

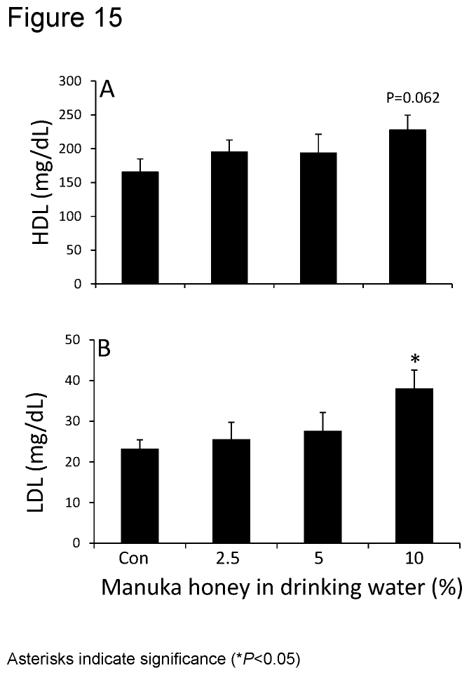

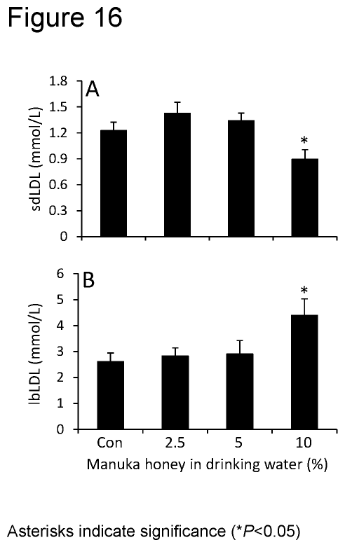

While concentrations of HDL and LDL cholesterol were increased in mice with manuka honey in their drinking water (FIGS. 15A and B), concentrations of small, dense LDL (sdLDL) were reduced (FIG. 16A), while those of large, buoyant LDL (lbLDL) were increased (FIG. 16B). The particle size of LDL cholesterol is related to atherogenicity with the smallest (sdLDL) being the most atherogenic and the lbLDL less atherogenic (Ivanova et al., 2017). sdLDL and lbLDL are markers for cardiovascular disease.

A modified method to that of Hirano et al was used to measure sdLDL, wherein 10 μL of plasma was added to 10 μL of 100 mmol/L MgCl₂ and 0.4% dextran sulfate (final concentration 50 mmol/L MgCl₂ and 0.2% dextran sulfate). The mixture was incubated at 37° C. for 10 min, then rested on ice for 10 min before centrifuging at 12000×g for 10 min to pellet VLDL, lbLDL and intermediate LDL. sdLDL and HDL remain in the supernatant. Therefore, concentrations of sdLDL could be measured in the supernatant using a mouse LDL-cholesterol assay ELISA kit (Crystal Chem #79980). Concentrations of lbLDL were calculated as the difference between total LDL and sdLDL.

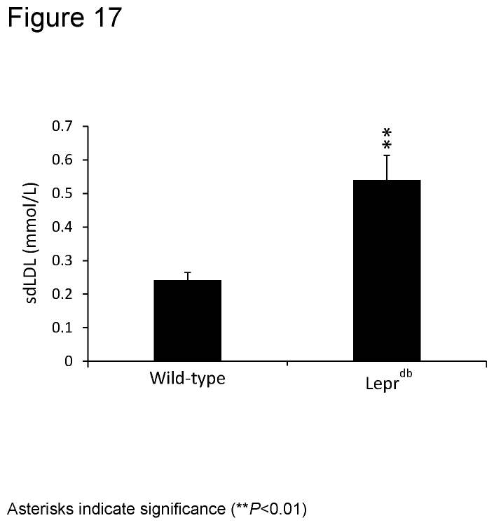

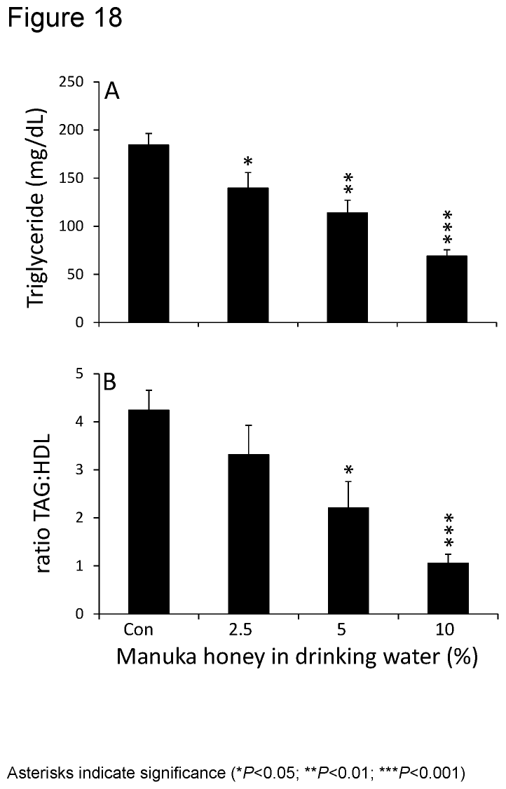

To confirm that concentrations of sdLDL were increased in obese, diabetic mice, compared with lean control mice, concentrations of sdLDL were measured in wild-type and lepr^{db} mice of the same age (18 weeks, n=5 per group) (wild-type were C57BL/6J, stock number 000664 and Lepr^{db} were stock number 000697, JAX Labs, ME, USA). Concentrations of sdLDL were increased in Leprdb mice compared with control (FIG. 17). Therefore, concentrations of sdLDL were increased in obese, diabetic mice. It was concluded that manuka honey can assist in achieving a healthier cholesterol profile.

A substantial dose-dependent decrease in concentrations of triglycerides was observed for mice that consumed manuka honey in their drinking water (FIG. 18A), and a similar decrease was observed in the ratio of triglycerides to HDL (FIG. 18B). The ratio of triglycerides to HDL is a useful clinical marker of atherogenicity because it correlates with the particle size of LDL (Boizel et al., 2000).

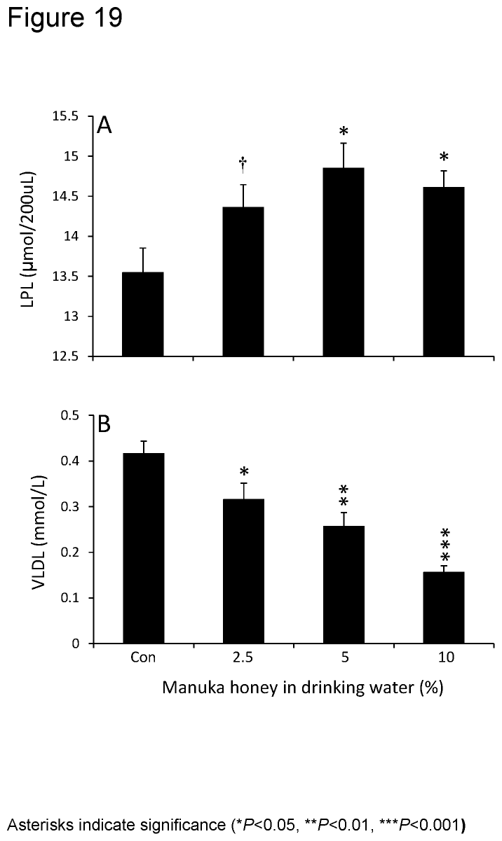

Chylomicrons and very low-density lipoprotein (VLDL) are triglyceride rich lipoproteins (TRLs) that are either degraded in the liver, or in the case of VLDL can be converted to LDL in plasma after liberation of triglyceride. The loss of triglyceride (lipolysis) requires the action of lipoprotein lipase (LPL). The inventors found that manuka honey increases the activity of LPL in a dose-dependent manner in blood (FIG. 19A), which corresponds to a decrease in concentrations of VLDL in blood (FIG. 19B) of mice.

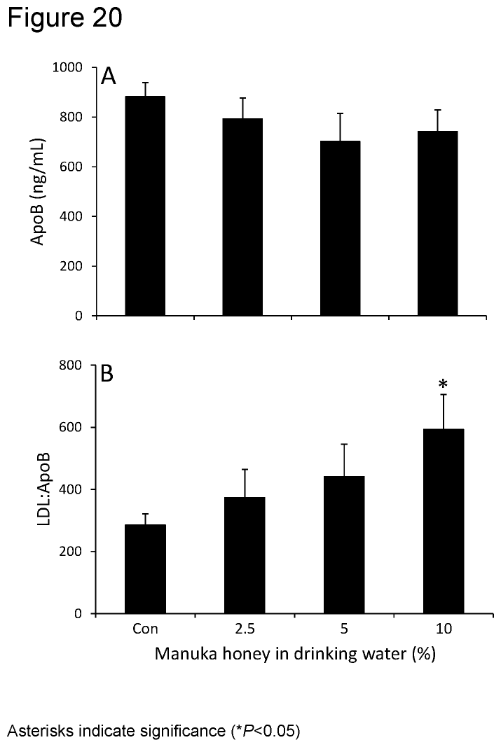

To further test for an increase in the particle size of LDL cholesterol, ApoB was measured and shown to be unchanged in blood (FIG. 20A). There is one ApoB protein per LDL cholesterol particle and, therefore, ApoB reflects the number of LDL particles. The increased ratio of LDL:ApoB in mice drinking manuka honey in their drinking water is consistent with an increase in the size of LDL particles (FIG. 20B).

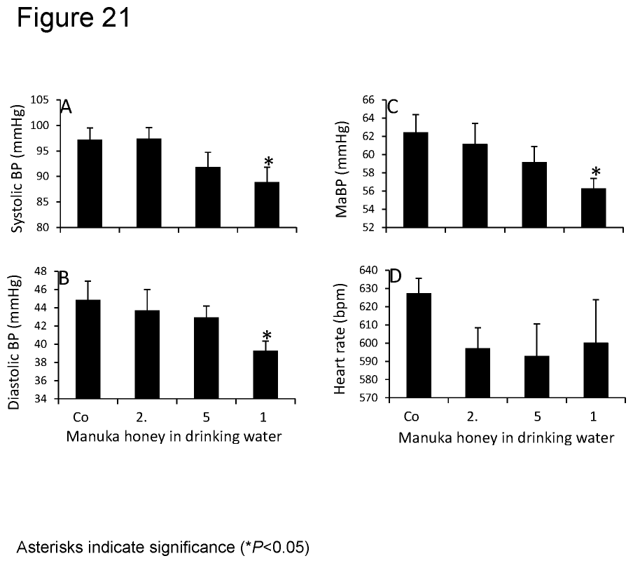

Lepr^{db} mice that drank water with manuka honey also had reduced systolic, diastolic and mean arterial blood pressure (FIG. 21), which is consistent with the effect of 125mer in this strain of mice.

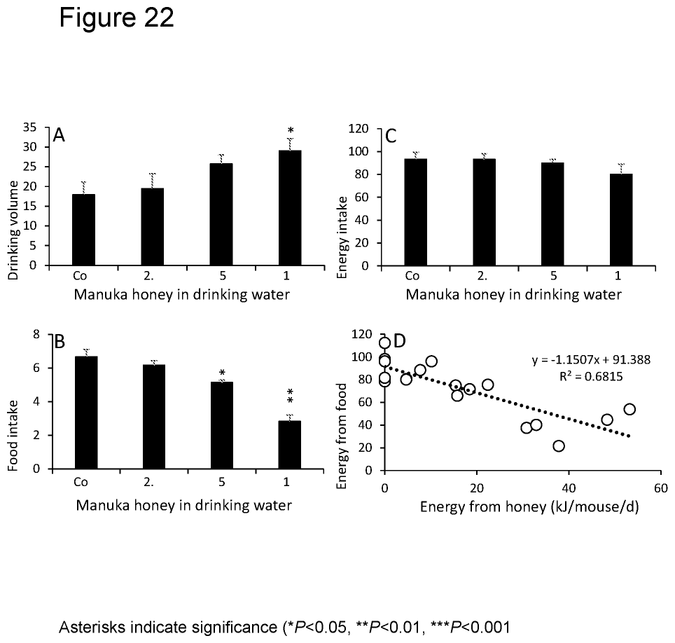

While there was a dose-dependent decrease in food intake, mice consuming manuka honey in their drinking water had an increased intake of fluid, which gave an equal intake of energy across all groups (FIG. 22). Therefore, the effect of manuka honey cannot be attributed to reduced energy intake

The inventors found that manuka honey does not exacerbate the diabetic condition of Lepr^{db} mice, or induce diabetes in lean mice despite the additional intake of sugar. Rather, manuka honey improves insulin sensitivity, reduces concentrations of HbA1c and improves the lipid profile. In particular, manuka honey reduces concentrations of triglycerides and sdLDL to create a healthier lipid profile. We attribute these benefits of manuka honey to the 125mer forming part of MRJP3 in the honey. The inventors found that the consumption of manuka honey and, thereby, MRJP3, is a means to improve the lipid profile in blood without compromising glucose homeostasis.

Human Doses

The general benefits experienced in the above trials with mice can be extrapolated to humans and other mammals. The dose of the 125mer injected into the mice converts to an adult human oral dose of 156 g of manuka honey per day (Table 2). In this regard the 125mer is provided by way of MRJP3 in the honey. The conversion method is documented in Reagan-Shaw et al (Reagan-Shaw et al., 2008). It assumes that the protein content of manuka honey is 0.25% by weight, and that MRJP3 is 18% of total protein concentration (Table 1).

TABLE 1
Proportion of detectable MRJP proteins (mean ± SD %) in manuka
honey (n = 12 samples) and royal jelly (n = 8 samples) as a percent
(by weight) of the total protein detected by SWATH-MS.

| Protein | Manuka honey | Royal Jelly |
|---|---|---|
| MRJP1 | 22.1 ± 1.3 | 32.7 ± 3.4 |
| MRJP2 | 14.6 ± 0.6 | 12.5 ± 1.2 |
| MRJP3 | 17.7 ± 1.1 | 14.3 ± 1.9 |
| MRJP4 | 2.1 ± 0.6 | 2.0 ± 0.4 |
| MRJP5 | 11.6 ± 1.3 | 15.0 ± 1.3 |
| MRJP6 | 4.5 ± 0.3 | 1.7 ± 0.4 |
| MRJP7 | 7.5 ± 0.9 | 5.9 ± 0.2 |

TABLE 2
Amount (g) of manuka honey required to be consumed for a
given concentration of protein to get a minimum
of 15 mg and a maximum of 70 mg of MRJP3 per day.

| Total | | Mass (g) of manuka honey to consume | | | | | |
|---|---|---|---|---|---|---|---|
| protein | MRJP3 | for a given percent of protein | | | | | |
| (mg) | (mg) | 0.25% | 0.5% | 0.75% | 1% | 2.5% | 5% |
| 83 | 15 | 33 | 17 | 11 | 8 | 3 | 2 |
| 167 | 30 | 67 | 33 | 22 | 17 | 7 | 3 |
| 222 | 40 | 89 | 44 | 30 | 22 | 9 | 4 |
| 278 | 50 | 111 | 56 | 37 | 28 | 11 | 6 |
| 333 | 60 | 133 | 67 | 44 | 33 | 13 | 7 |
| 389 | 70 | 156 | 78 | 52 | 39 | 16 | 8 |

The dose of manuka honey consumed by the mice in drinking water (5% and 10% w/v) equates to an adult human oral dose of 33-67 g of manuka honey per day. This assumes a protein content of 0.25% by weight and is based on the conversion method of Reagan-Shaw et al (Reagan-Shaw et al., 2008) (see Table 2). It also equates to an oral dose of full-length MRJP3 of 15 mg-70 mg per day.

The mice were not offered more than 10% of manuka honey in their drinking water because of the high sugar content of honey (70%), which may have been problematic for the Lepr^{db} strain (obese and diabetic)

Enhancement of Honey with Royal Jelly

The protein concentration of honey is relatively low, being 0.058-0.786% by weight (White and Rudyi, 1978). That content, including the content of MRJP3 and, therefore, the 125mer, may be increased for the above therapeutic uses by adding royal jelly. In this regard royal jelly is approximately 13% by weight protein (Pavel et al., 2013). It provides a means for naturally increasing the protein concentration of manuka honey, preferably by 0.3-3% by weight. Adding royal jelly reduces the amount of honey that needs to be ingested, without compromising on the amount of MRJP3, and therefore the amount of the 125mer, received. Table 2 illustrates the proportional decrease in the volume of manuka honey needed to provide 15 mg or more of MRJP3 per day. The preferred amount is between 30 and 70 mg per day, which for a manuka honey batch that has 0.75% protein would be a volume between 22 and 52 g (Table 2).

Method of Enhancing the Protein Content of Manuka Honey

Honey has a lower concentration of protein than royal jelly (0.17 vs 13%) (White and Rudyi, 1978, Pavel et al., 2013). Therefore, one means to increase the concentration of total proteins, which includes MRJP3, in honey is to add royal jelly to manuka honey. Described herein is a method for mixing royal jelly into manuka honey to increase the concentration of total protein and, in particular, the concentration of MRJP3. We also describe herein a method of use wherein the mixture of royal jelly and manuka honey is consumed to lower concentrations of triglycerides, LDL and sdLDL cholesterol, insulin, glucose, HbA1c and to increase insulin sensitivity and to lower blood pressure.

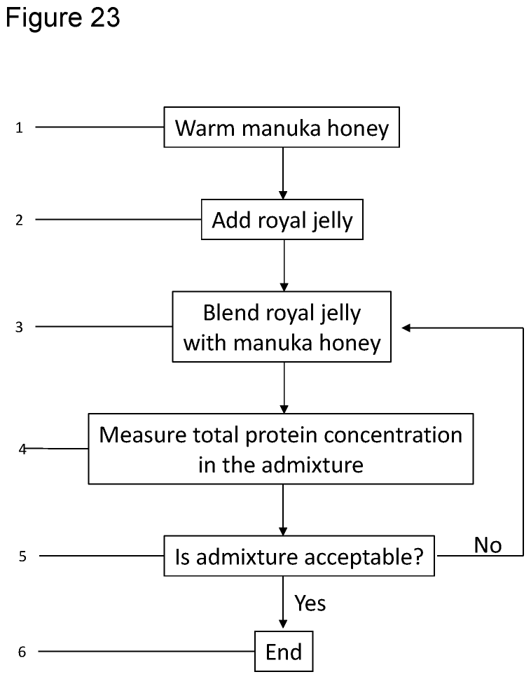

FIG. 23 illustrates a method of mixing royal jelly into manuka honey. In the first step the honey is heated to 25-45° C. (most preferably 35-42° C.) within a holding tank having built in mixers that may include paddles. In the second step royal jelly is added and in the third it is blended into the honey. The protein content of the mixture is measured in the fourth step. If the content meets the desired level then no further blending is needed, otherwise more royal jelly is added and blended, and the test run again. Testing, adding royal jelly and blending are repeated, if necessary, until the mixture has the desired protein content, preferably 0.3-3% by weight, and most preferably 0.3-5% by weight.

As the percent of MRJP3 does not differ greatly across batches (Table 1), a standard protein assay is used, for example as per Bradford, Lowry, Bicinchoninic (BCA), or similar, to measure the concentration of total protein. From the concentration of total protein, the concentration of MRJP3 can be calculated based on there being 17.7% by weight of total protein in manuka honey and 14.3% by weight of protein in royal jelly (Table 1).

In a further embodiment of the invention MRJP3 protein made by recombinant or synthetic means, is added to manuka honey instead of, or as well as, royal jelly. The process of mixing and testing is otherwise the same as above.

In terms of disclosure, this document hereby discloses each item, feature or step mentioned herein in combination with one or more of any of the other item, feature or step disclosed herein, in each case regardless of whether such combination is claimed.

While some preferred embodiments of the invention have been described by way of example it should be appreciated that modifications and improvements can occur without departing from the scope of the following claims.

1. Use of a peptide or fragment thereof comprising SEQ ID NO. 1, or a peptide or fragment having at least 80% sequence identity thereto, in the manufacture of a medicament for one or more of:
a) reducing triglycerides;
b) reducing LDL;
c) reducing sdLDL;
d) reducing glucose;
e) reducing insulin;
f) reducing HbA1c; and
g) increasing insulin sensitivity.

2. A use according to claim 1, wherein the peptide or fragment has at least 85% sequence identity to SEQ ID NO. 1.

3. A use according to claim 1, wherein the peptide or fragment has at least 90% sequence identity to SEQ ID NO. 1.

4. A use according to claim 1, wherein the peptide or fragment has at least 95% sequence identity to SEQ ID NO. 1.

5. A use according to claim 1, wherein the peptide or fragment has at least 99% sequence identity to SEQ ID NO. 1.

6. A use according to claim 1, wherein the peptide or fragment has 100% sequence identity to SEQ ID NO. 1.

7. A use according to claim 6, wherein the SEQ ID NO. 1 is within MRJP3.

8. A use according to claim 6 or 7, wherein the SEQ ID NO. 1 is within honey.

9. A method of treatment, comprising administering or taking a peptide or fragment thereof, comprising SEQ ID NO. 1, or a peptide or fragment having at least 80% sequence identity thereto, to:
a) reduce triglycerides;
b) reduce LDL;
c) reduce sdLDL;
d) reduce glucose;
e) reduce insulin;
f) reduce HbA1c; and
g) increase insulin sensitivity.

10. A method according to claim 1, wherein the peptide or fragment has at least 85% sequence identity to SEQ ID NO. 1.

11. A method according to claim 1, wherein the peptide or fragment has at least 90% sequence identity to SEQ ID NO. 1.

12. A method according to claim 1, wherein the peptide or fragment has at least 95% sequence identity to SEQ ID NO. 1.

13. A method according to claim 1, wherein the peptide or fragment has at least 99% sequence identity to SEQ ID NO. 1.

14. A method according to claim 1, wherein the peptide or fragment has 100% sequence identity to SEQ ID NO. 1.

15. A method according to claim 14, wherein the SEQ ID NO. 1 is within MRJP3.

16. A method according to claim 14 or 15, wherein the SEQ ID NO. 1 is within honey.

17. The use of MRJP3, or honey containing MRJP3, in the manufacture of a medicament for one or more of:
a) reducing triglycerides;
b) reducing LDL;
c) reducing sdLDL;
d) reducing glucose;
e) reducing insulin;
f) reducing HbA1c; and
g) increasing insulin sensitivity.

18. A method of treatment comprising administering or taking MRJP3, or honey containing MRJP3, to:
a) reduce triglycerides;
b) reduce LDL;
c) reduce sdLDL;
d) reduce glucose;
e) reduce insulin;
f) reduce HbA1c; and
g) increase insulin sensitivity.

19. A method according to claim 18, wherein the honey is manuka honey.
